(19) United States
(12) Patent Application Publication
(10) Pub. No.: US 2015/0065916 A1
(43) Pub. Date: Mar. 5, 2015

(54) FULLY AUTOMATED VASCULAR IMAGING AND ACCESS SYSTEM

(71) Applicant: VascuLogic, LLC, Piscataway, NJ (US)

(72) Inventors: Tim Maguire, Piscataway, NJ (US); Alvin Chen, Holmdel, NJ (US)

(73) Assignee: VascuLogic, LLC, Piscataway, NJ (US)

(21) Appl. No.: 14/013,181

(22) Filed: Aug. 29, 2013

(51) Int. Cl. A61B 19/00; A61B 5/15

(52) U.S. Cl. CPC A61B 19/2203; A61B 19/5244; A61B 5/150206; A61B 2019/2207; A61B 2019/5246
USPC 600/573; 606/130

(57) ABSTRACT

The present invention is directed to a self-contained, fully automated vascular imaging and access device, methods of imaging, mapping, and analyzing three-dimensional views of blood vessels, and methods for providing continuous and real-time communication with a robotically actuated needle and a computer interface.

### BACKGROUND OF THE INVENTION

The first step in many clinical interventions is to establish venous or arterial access to the bloodstream, a procedures that is critical for a wide range of clinical applications, including blood draws, diagnostic tests, and the delivery of medication, nutrients, and other fluids through the bloodstream.

Currently, venipunctures are executed manually by trained personnel, but there are problems inherent with these processes. Successful placement of the cannula requires the coordination of complex deductive and visuomotor tasks in real time. Many times locating a vein or artery is a challenge, especially in young, elderly, dark-skinned, and obese patients whose veins are small, fragile, rolling, or obscured by melanin or subcutaneous fat. To complicate matters, multiple attempts at needle insertion may be required, due either to the inexperience of the person obtaining the sample, and/or from difficulty in locating the target vessel. Success depends heavily on the procedure, patient type, and clinician's experience, and mistakes result in multiple punctures, therapy delays, adverse effects, pain, and stress for the patient, the family, and the medical staff.

Venipuncture is furthermore the leading cause of bloodborne pathogen transmission among US healthcare workers. Annual reports estimate 600,000-800,000 accidental needlestick injuries in the U.S., a percentage of which can lead to serious or fatal infections.

In total, failed vessel punctures are estimated to cost the U.S. health care system $4.7 billion/year.

Traditionally guided by visual inspection and manual palpation, successful placement of the cannula into the vein requires the coordination of complex deductive and visuomotor tasks in real time. Unfortunately, the extents of training, experience, and skill vary widely between healthcare personnel.

To aid in locating target veins for venipuncture, some companies have commercialized systems using imaging techniques—two examples are the Luminetx's VeinViewer and the AccuVein AV300. These devices detect subcutaneous veins and project images back on the skin, providing a two-dimensional (2D) positional guide for venipuncture. Although this technology may provide methods of viewing veins externally, it does not provide any depth representation of the veins under the skin, is frequently ineffective for difficult venous access patients, and does not address the frequency of needle-induced bloodborne pathogen transmission among practitioners. These technologies are furthermore limited to peripheral cannula insertions as they lack the imaging depth needed to visualize deeper central vessels.

Acoustic ultrasound imaging systems have also been developed to provide 3D representation of subcutaneous blood vessels and assist clinical practitioners in cannula insertion. Although ultrasound based technologies have the advantage of providing greater imaging depth to allow peripheral and central vessel access, the effective use of these technologies requires a substantial amount of clinical training and is frequently subject to human error.

In addition to aiding in vein viewing, there is much work into developing robotically-guided systems for needle insertion into organs for surgical purposes. Commercial robotic systems have been developed for prostatectomy using CT/MRI; other applications include orthopedic and neurosurgery, endoscopy, telesurgery, and animal research. Ultrasound-guided robots have also been developed for various applications. These robotic systems generally combine an image guidance method, analysis and control software, and a robotic effector to control the position of the needle or cannula. However, these systems are not conducive to providing automated venous or arterial access.

Despite significant advances in the fields of medical robotics and computerized surgery, currently there is no technology that is able to directly address the fundamental problems of patient and practitioner safety for vessel punctures. Furthermore there is no technology for complete automated cannula insertion for the purpose of access to peripherally or centrally located blood vessels. Finally there is no technology that combines an imaging system with a robotically driven for the purpose of blood vessel puncture.

### OBJECTS OF THE PRESENT INVENTION

It is an object of the present invention to develop a technology for fully automated vascular imaging and access.

It is another object of the present invention to provide a self-contained, compact, portable device to provide methods for withdrawing fluid from and introducing fluid into blood vessels.

It is another object of the present invention to provide a device that is adapted for multiple patient types, which may include but are not limited to neonatal patients, pediatric patients, elderly patients, patients of varying age, patients of varying skin tone, and patients of varying body weight.

It is another object of the present invention to provide a device that is adapted for multiple clinical environments, which may include but are not limited to emergency care, intensive care, first responders, ambulatory care, neonatal and pediatric care, inpatient care, outpatient care, walk-in clinics, and at-home care.

It is another object of the present invention to provide a device that will use one or more optical, acoustic, photoacoustic, tactile, or force-based methods for detecting subcutaneous vessels and enhancing these images with filtering techniques.

It is yet another object of the invention to non-invasively image and map in real time the three-dimensional (3D) spatial coordinates of subcutaneous vessels in order to robotically direct a needle into the optimal or designated vessel for a puncture at controlled vertical and horizontal injection angles and adjustable injection speeds.

It is yet another object of the invention to provide a device that utilizes image processing, image segmentation, image reconstruction, feature extraction, dimensionality reduction, statistical analysis, decision making/weighting, and/or vessel selection.

It is yet another object of the invention to provide a device that directs a robotically driven needle or cannula into a target vessel to provide accurate single-attempt vessel access.

It is yet another object of the present invention to provide an all-in-one point of care device for venipuncture and for providing simultaneous real-time diagnostic assays. Specifically, it is an object to provide methods for obtaining analytical assays such as glucose monitoring, pregnancy/ovulation testing, coagulation/PT evaluation, fecal occult blood, determination of drugs of abuse, detection of bacterial infections (e.g., H. pylori), detection of HIV, and monitoring of cholesterol levels utilizing the self-contained, automated venipuncture device.

In accordance with the above-mentioned objects, the present invention is directed to a self-contained, automated venipuncture device containing three major components: (1) an imaging system; (2) an automated robotic end-effector unit; and (3) a computer.

The present invention is further directed to an automated system for vascular imaging and access, comprising an imaging system for providing continuous and real-time imaging of blood vessels, said imaging system comprising at least one of optical, acoustic, photoacoustic, or tactile imaging; image processing software for generating a continuous and real-time three-dimensional (3D) computer model of the blood vessels based on the imaging system, and selecting an optimal vessel target based on visual and anatomical information for inserting a needle into the selected vessel target during a cannulation routine on a human subject; a robotic effector comprising a needle, a needle attachment unit, and a needle actuation system that positions the needle at the selected vessel target located by the image processing software and moves the needle toward and insert the needle into the selected vessel target; and a computer connected to the imaging system and robotic effector, said computer directing information continuously and in real-time to and from the imaging system, image processing software, and robotic effector in order to autonomously adjust the position and orientation of the needle with respect to the selected vessel target during the cannulation routine. In preferred embodiments, the robotic effector is adapted for placement on an appendage of a human or animal subject. Preferably, the robotic effector can withdraw fluid from the selected vessel target or deliver fluid through the selected vessel target. Preferably, the needle is affixed to the robotic effector through the needle attachment unit such that the needle may be attached or detached manually or automatically before or after the cannulation routine.

In certain embodiments, the imaging system is a portable or tabletop device, capable of either being mounted onto a subject's appendage or being placed onto a stationary unit. The imaging system preferably comprises at least one of: at least one light emitting source having a wavelength emission range within the visible or infrared spectrum and one or more optical detectors; or at least one ultrasound transducer, the acoustic signal being transmitted and received through the at least one ultrasound transducer and converted from analog to digital form for image formation and processing; or at least one visible or near infrared optical light source and at least one ultrasound transducer, the photoacoustic signal being transmitted through the at least one optical light source, received through the at least one ultrasound transducer, and converted from analog to digital form for image formation and processing. The imaging system preferably further allows for at least one of thermal imaging, spectroscopic imaging, diffuse optical tomography imaging, ultrasound Doppler imaging, ultrasound color Doppler imaging, 3D/4D ultrasound imaging, photoacoustic Doppler imaging, photoacoustic color Doppler imaging, or 3D/4D photoacoustic imaging. The imaging system is preferably adapted to illuminate structures in addition to blood vessels, the additional structures comprising one or more of arm length, arm thickness, anatomical markings, markings on the skin surface, signs of infection, signs of fluid leakage, or nerves. The imaging system is preferably further adapted to differentiate between veins and arteries, and preferably can be adjusted to maximize imaging quality on a per patient basis.

The automated system is preferably enhanced with at least one of diffusive filters, polarizing filters, spatial filters, coherence-based filters, wavelength or frequency-based bandpass filters, or electronic analog filters.

The imaging system is preferably contained within an imaging housing unit. The imaging housing unit may be temporarily disengaged from the robotic effector, the imaging system then being used as a standalone imaging system to visualize blood vessels or other structures. When the imaging system is contained within the imaging housing unit and when disengaged from the robotic effector, can remain in communication with the computer.

The image processing software preferably processes and maps a three-dimensional (3D) computer model of blood vessels continuously and in real-time, via one or more of the following: increasing the visibility of vessels or decrease the amount of artifacts and noise in determining an optimal vessel target for cannulation; computing the depth of the vessels and building a three-dimensional model of the vessels; determining an the optimal vessel target for cannulation and the optimal needle orientation relative to a the-selected vessel target; tracking the position and orientation of a the selected vessel target and the needle tip as the needle is moved toward and inserted into the selected vessel target; relaying information about the position and orientation of the selected vessel target and the needle based on the vessel and needle tip positions as the needle is moved toward and inserted into the selected vessel target.

The detected veins are preferably automatically labeled and the selected vessel target is automatically determined based on at least one of the size and anatomical structure of the blood vessels, the quality of the image at each location, the particular needle and application, and the subject's medical information.

The 3D position of the target vein and the 3D position of the needle tip may be computed in real time, and the relative distance between the 3D positions of the target vein and the needle tip may be computed as the needle is inserted, and the needle insertion is preferably halted when the distance is zero. The pose of the needle is preferably computed in real time, and the correct angle of injection is preferably ensured utilizing fine motor positioning adjustments. Preferably, at least one of optical, mechanical, magnetic, or potentiometric sensors are coupled to at least one motor actuator in order to collect signals continuously and in real-time to indicate the rotational position and motion of the at least one motor actuator. Preferably, at least one force sensor is coupled to the needle in order to collect signals continuously and in real-time to indicate mechanical forces acting on the needle as the needle is inserted.

The feedback guidance methods preferably additionally comprise a computer model of the mechanical interactions between the needle and the skin tissue, the computer model taking in signals from at least one feedback sensor and outputting the position of the needle tip and the target vessel continuously and in real-time.

Preferably, the robotic effector is capable of at least two of the following degrees of motion: i) horizontal motion along the length of the subject's appendage, ii) horizontal motion across the width of the subject's appendage, iii) vertical motion relative to the surface of the subject's appendage, iv) vertical angular rotation of the needle, v) horizontal angular rotation of the needle, vi) rotation of the needle along its axis, and vii) forward motion of needle insertion.

In certain preferred embodiments, wherein at least two motors are incorporated independently in order to control the movement of the needle and provide fine adjustment of the needle pose. In other preferred embodiments, at least three motors are incorporated into a kinematic chain or robotic arm in order to control the movement and orientation of the needle and provide fine adjustment of the needle pose.

In certain preferred embodiments, real-time video images are provided to the computer, the images being stored in digital format on a digital storage device connected to or inside of the computer. Preferably, a user interface is included wherein at least one of the continuous and real-time images or the computer model of blood vessels is displayed digitally on a screen. The anatomical labels of structures within the images or computer model may be additionally displayed on the screen. Further, input settings and parameters can be controlled manually prior to or during the cannulation routine in order to direct the cannulation routine.

In certain embodiments, a vessel target can be selected manually and compared to the automatically selected optimal vessel target. In additional embodiments, the cannulation routine can be manually terminated. The movement of the subject's appendage may be reduced by securing the appendage in position prior to inserting the needle. Thus, in certain embodiments further comprises tightening over the subject's appendage, wherein the tightening process can additionally serve as providing a tourniquet. The system may additionally comprise preventing the subject's appendage from coming in contact with the remaining components of the automated system. Further, the system is preferably adaptable to different patient sizes.

The components of the system preferably may be disengaged for the purpose of at least one of cleaning, sterilization, maintenance, or interchanging with other systems.

The automated system may be further adapted to provide analytical or diagnostic testing, comprising obtaining a fluid sample utilizing the self-contained, automated system and introducing the fluid sample into the test and/or interchanging different tests for a single patient or in between use by different patients.

The present invention may furthermore use tactile imaging to detect vessels and differentiate veins and arteries prior to injection, as well as to provide real-time tracking, monitoring and detection during injection. An example may include, but is not limited to, using load cells coupled with the needle to detect axial and normal friction forces on the needle. A second example may include pressing a piezoresistive, capacitive, or conductive pressure sensor pads onto the skin surface to detect pressure changes that indicate the presence of superficial vessels directly beneath the skin. The tactile feedback system can also be extended to also provide pressure on the injection site before, during, or after the cannulation.

The present invention may use one or multiple imaging modalities detect subcutaneous vessels, differentiate between veins and arteries, and track the position of the needle.

The imaging systems can be enclosed within an imaging housing unit. The imaging housing unit may be disengaged from the remainder of the device to be used as a standalone imaging system.

The imaging system and end-effector unit can additionally be contained in a single unit. This unit will be capable of either being mounted onto a target limb (classically the forearm for venipuncture) of the subject or the target limb will be placed onto a stationary unit (e.g., a table). The imaging system and end-effector unit can be remotely connected to a computer which controls the image processing and robotic automation.

The device can use a number of image analysis, feature extraction, 3D reconstruction, dimensionality reduction, scoring, and decision-making techniques to control the automated injection at the proper cannulation location.

Using one or more imaging techniques, subcutaneous vessels can be visualized and a three-dimensional map of major vessels constructed on a computer. Using both instant and real time coordinates generated by the imaging system and image processing software, the robotically controlled needle can be guided into a target vessel.

The device can incorporate methods to support the subject's appendage or limb during the cannulation routine. By providing a support, the device can improve the comfort of the procedure and reduce movement. Numerous methods for providing support may be utilized, for example an air-inflatable cuff-like structure enclosed around the appendage, similar in nature to a blood pressure cuff, that is adapted to different patient sizes. The support may furthermore be tightened either above or below the site of cannulation, to serve as a tourniquet. Finally, all components of the support that may come in contact with the subject may be disengaged from the remainder of the device for the purpose of sterilization.

The self-contained, automated venipuncture device of the present invention can also be coupled with real-time diagnostic assays, to provide an all-in-one point of care device.

The creation of three-dimensional (3D) coordinate representation of superficial vessels in a rapid and real time manner eliminates any guess work and allows precise needle insertion. The self-contained, automated venipuncture device of the present invention therefore eliminates human error and potential multiple and incorrect punctures that are common occurrences when performing a venipuncture, each of which can cause trauma and painful bruising for the human. The device furthermore eliminates the frequent cases of bloodborne disease transmission suffered by practitioners from regularly working with contaminated needles.

The methods utilized in the present invention can increase patient comfort, practitioner safety, and overall hospital efficiency for extremely common procedures, all of which are priority in health care.

### BRIEF DESCRIPTION OF THE DRAWINGS

FIG. 1A shows a self-contained, automated venipuncture device of the present invention connected to a human and hard wired to the computer (PC) interface component of the device.

FIG. 1B is a close-up of a self-contained, automated venipuncture device of the present invention.

FIGS. 2A and 2B illustrate an underside layout of an imaging system housing plate and the assembly of imaging system components.

FIG. 3A is an illustration a cuff-like harness, automated robotic end-effector unit and imaging system.

FIG. 3B shows an automated robotic end-effector “positioning” unit and its individual robotic components.

FIG. 3C illustrates an imaging system assembly in association with the robotically driven needle of the automated robotic end-effector unit.

FIG. 4 is a flow diagram of computations made to reconstruct a three-dimensional (3D) representation of a vein from two-dimensional (2D) images.

FIG. 5 is a flow diagram of a vessel puncture control algorithm and the methods for the control of the venipuncture device.

FIG. 6 shows an Example Decrease in force observed after needle has penetrated a target vein.

FIG. 7 is a graph showing the wavelength absorptivity of deoxy-HB and oxy-Hb.

FIG. 8 is a diagram showing one possible configuration of light emitting diodes and light detectors of the imaging system.

FIG. 9A is a schematic of the ultrasound imaging configuration.

FIG. 9B is a schematic of the photoacoustic imaging configuration.

FIG. 10 is a schematic of the force feedback system.

FIG. 11 is a flow diagram of the automated vessel selection and tracking algorithm.

### DETAILED DESCRIPTION OF THE INVENTION

The invention is directed in part to a fully automated system for peripheral vein imaging and access. The system preferably includes an imaging system for providing continuous and real-time imaging of blood vessels, said imaging system comprising at least one of optical, acoustic, photoacoustic, or tactile imaging. The automated system further preferably comprises image processing software for generating a continuous and real-time three-dimensional (3D) computer model of the blood vessels based on the imaging system, and selecting an optimal vessel target based on visual and anatomical information for inserting a needle into the selected vessel target. The system further comprises a robotic effector comprising a needle, a needle attachment unit, and a needle actuation system that positions the needle at the selected vessel target located by the image processing software, and a computer connected to the imaging system and robotic effector, said computer directing, continuously and in real-time, information to and from the imaging system, image processing software, and robotic end-effector in order to autonomously adjust the position and orientation of the needle with respect to the selected vessel target during the cannulation routine.

### Imaging System

The automated vessel puncture device of the present invention contains a three-dimensional (3D) imaging system that non-invasively maps the target veins, for example a subcutaneous network of blood vessels.

The three-dimensional (3D) imaging system of the present invention contains one or more signal sources and one or more detectors for vessel visualization. Methods of imaging include visible or infrared optical imaging, acoustic imaging, photoacoustic imaging, and tactile and force based imaging. A combination of these methods can also be utilized, and in one preferred embodiment optical, acoustic, and tactile imaging modalities are combined and integrated with the computer and robotic effector.

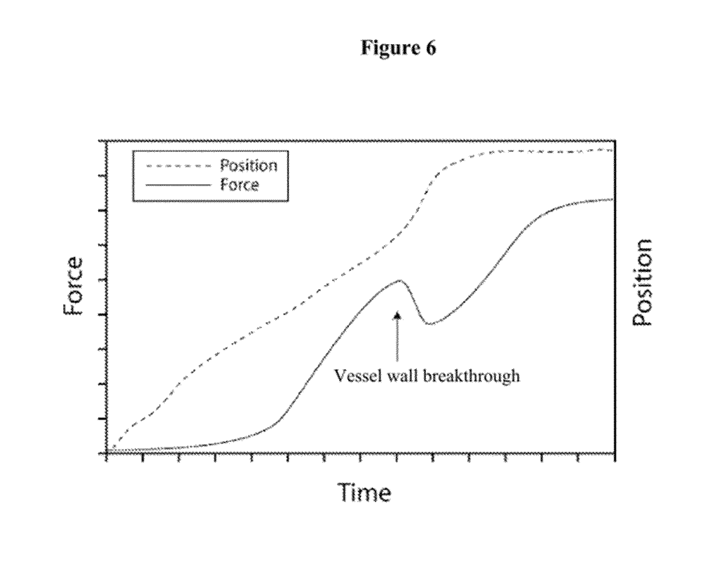

The optical imaging system utilize visible and infrared light to illuminate blood vessels to within 4 mm depth of penetration. In one preferred embodiment, multiple light emitting diodes (LEDs) or laser diodes are utilized in the imaging system as the signal source. In the case of visible light imaging, superficial vessels between 0.5 and 1.5 mm in depth is emphasized. In the case of infrared imaging, vessels lying between 1.5 and 4 mm are emphasized, and the wavelength range exists between about 730 nm to about 1060 nm. In certain preferred embodiments, the light emitting diodes have a wavelength ranging from about 850 nm to about 950 nm, these wavelengths representing local peak absorptions of deoxy-Hb (See: FIG. 7).

Light emitting diodes (LEDs) of other frequencies are also contemplated for use in the imaging system of the present invention.

In certain other embodiments, the present invention may utilize other sources of light, such as far infrared or thermal imaging.

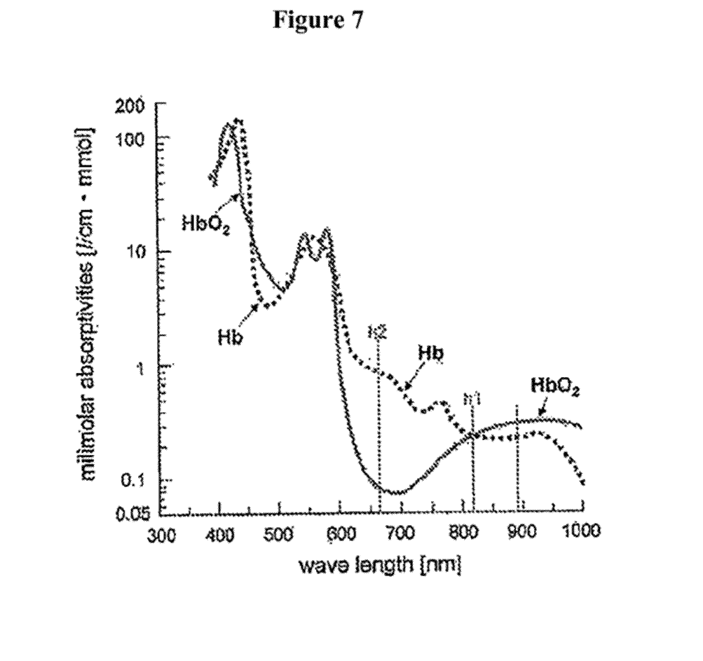

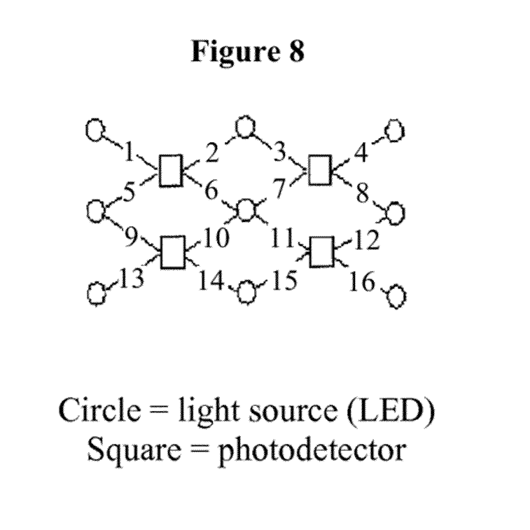

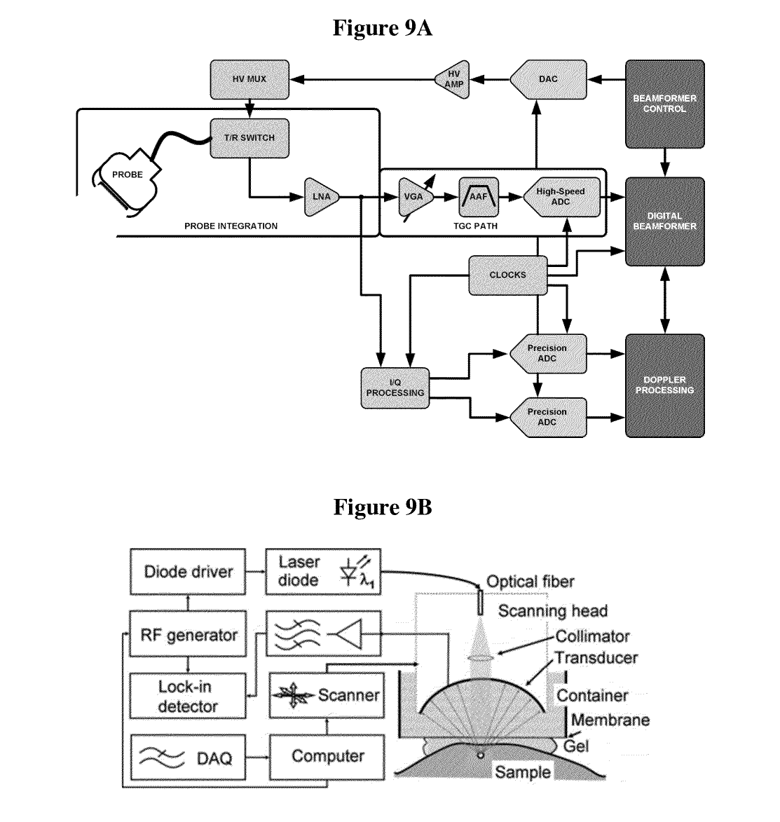

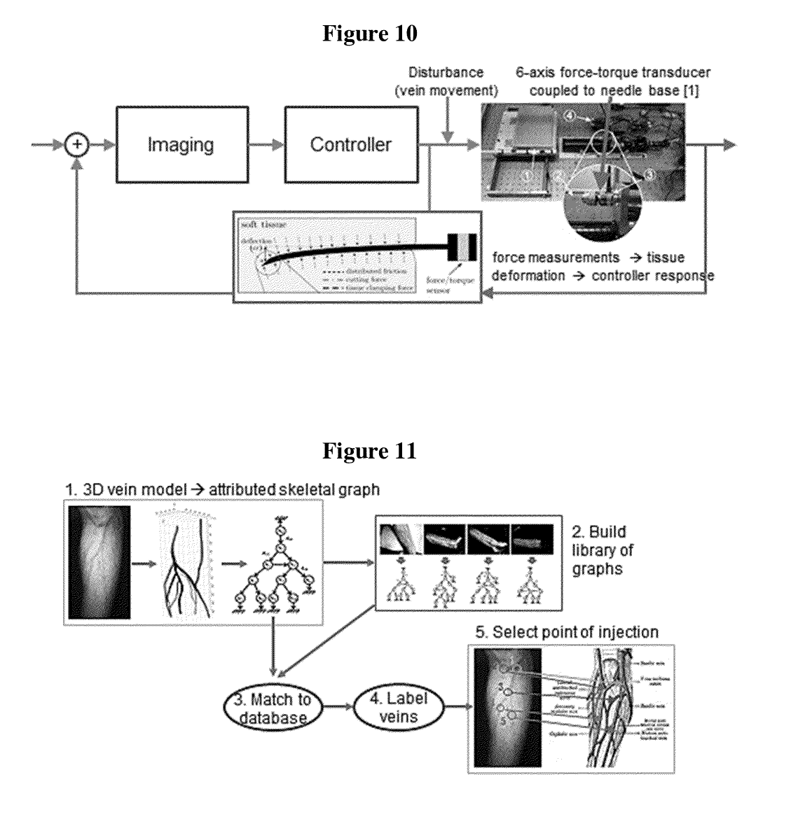

In certain embodiments of the present invention, the near infrared light emitting diodes are arranged in an array that preferably also includes the detectors. One possible configuration is as shown in FIG. 8, wherein the circles represent the LEDs and the squares represent the detector.

In this embodiment, there are nine (9) light emitting diodes, each capable of generating light at either 760 nm or 940 nm, and four (4) photodetectors, resulting in 16 possible LED-detector pairings, each corresponding to a possible orientation of the subcutaneous vessels. The raw measurement from the imaging system is:

D=(ƒ_{deoxy,1}−ƒ_{oxy,1})−(ƒ_{deoxy,2}−ƒ_{oxy,2})

where 1 and 2 refer to two different LEDs. The quantity D is a vector, having a magnitude representing the difference of differential absorption and a direction representing any two of the paths enumerated above. This particular embodiment is not intended to be limiting in any way.

During the imaging process, the Dᵢ measurements are collected and then used to reconstruct the position and orientation of the subcutaneous vein. Back-projection techniques similar to traditional computed tomography are then used to compute the 3D reconstruction. With this data, a 3D coordinate system can be generated that will be used to automatically guide the robotically driven needle.

In certain other embodiments, near infrared sensitive charged-coupled device (CCD) or CMOS cameras may be used to detect reflected light, in combination with specific filters to enhance the signal.

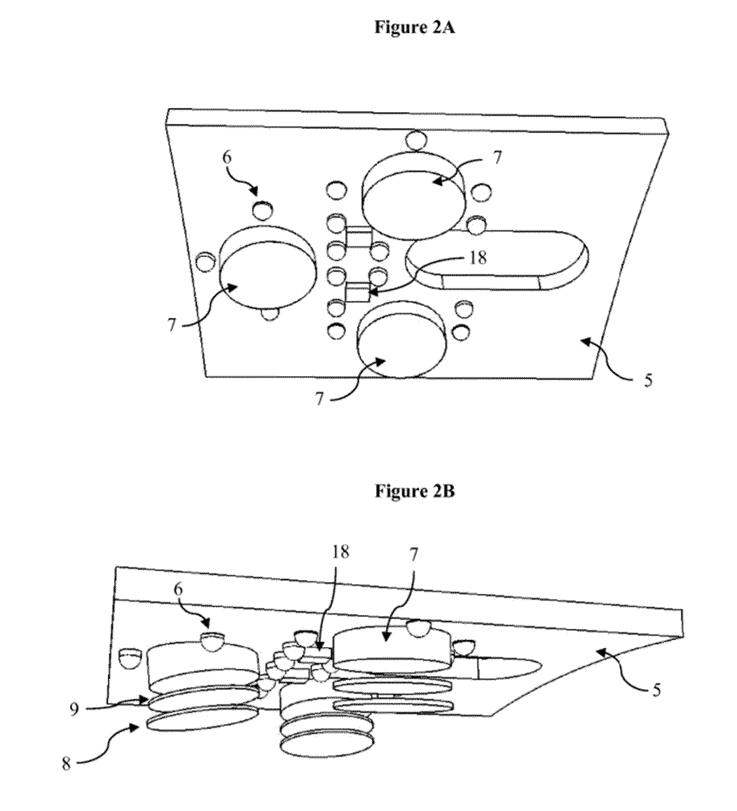

FIGS. 2A and 2B show an example of the components of the imaging system of the self-contained, automated venipuncture device of the present invention. The imaging system shown in FIGS. 2A and 2B contains a plurality of highly diffuse infrared light emitting diodes (LEDs) 6 for illuminating the target vessel of a human, such as a vein. The imaging device further contains a plurality of light detectors 7 for capturing an image, e.g., video image, of the target veins based upon infrared light reflected from the vessel. When the target vessels are disposed below subcutaneous fat in body tissue, the vessels can be clearly seen in a video image produced by the imaging system.

The light emitting diodes 6 may be “potted” or surrounded on their sides by a substantially opaque material which minimizes diffusion of light from the side of the light emitting diode 6. For optimum illumination, each light emitting diode should be focused at a select angle to maximize the concentration of light source at a select location within the target vessel. For example, a 15 degree angle of dispersion (or focus angle) may be utilized for effective illumination. In certain embodiments, a dispersion angle of 30 degrees may be suitable for effective illumination. Other angles of dispersion (or focus angles) may be acceptable as well. The relatively narrow focus angle is beneficial as more light is directed into the human's tissue around the target vessel for trans-illumination. Each of the light emitting diodes 6 can be secured to an imaging system housing plate 5. The imaging system housing plate 5 is preferably a printed circuit board with integrated contacts for connecting to a battery source.

The imaging system of the present invention may also contain on the imaging system housing plate 5 a position sensor 18, as seen in FIGS. 2A and 2B, for providing a measurement of distance from the light emitting diodes 6 and the needle 13 to the target veins. This will ensure exact distances are known at all times. The position sensor located 18 on the imaging system may utilize a laser based system to determine the distance between the device and the target vein. The position sensor 18, together with the computer program sends out a burst/ping of laser light and determines the time for the laser light to bounce back. This time is then correlated with a distance.

As further shown in FIGS. 2A and 2B, the imaging system may also contain a plurality of infrared filtered light detectors 7. FIG. 2B further shows each light detector 7 containing a filter 8 on the lens 9 of the light detector that will allow only those wavelengths in the infrared range to pass through and then be subsequently imaged. The imaging system of the present invention may utilize interchangeable lenses to vary the field of view of the light detectors 7. The filter 8 and lens 9 setup may be attached to an image acquisition assembly unit of the light detector 7, through an attachment to the light detector 7.

The lens 9 may be configured to further focus the light emitted from the light emitting diodes 6 as desired. Alternatively, the lens 9 may be a variable focusing lens that is extended or retracted relative to a cylindrical extension to vary the focus of the light emitting diodes 6.

In certain embodiments of the present invention, the light detectors 7 will also be capable of being fixed or mounted on a motorized platform to pivot, providing image acquisition from various angles.

Use of the light emitting diodes 6 as a light source minimizes the danger of burning patients with whom the self-contained, automated venipuncture device is used and will prevent injury to the eyes of a clinician or the human if they inadvertently look directly into the light source. The lens 9 further shields the human from any heat which is produced by the light emitting diodes 6. As previously discussed, light emitting diodes 6 are available which emit in a relatively narrow spectral band, preferably with a predominant wavelength of about 700 nm to about 910 nm. Light with this wavelength has been found to highlight target vessels, e.g., veins, with respect to the tissue.

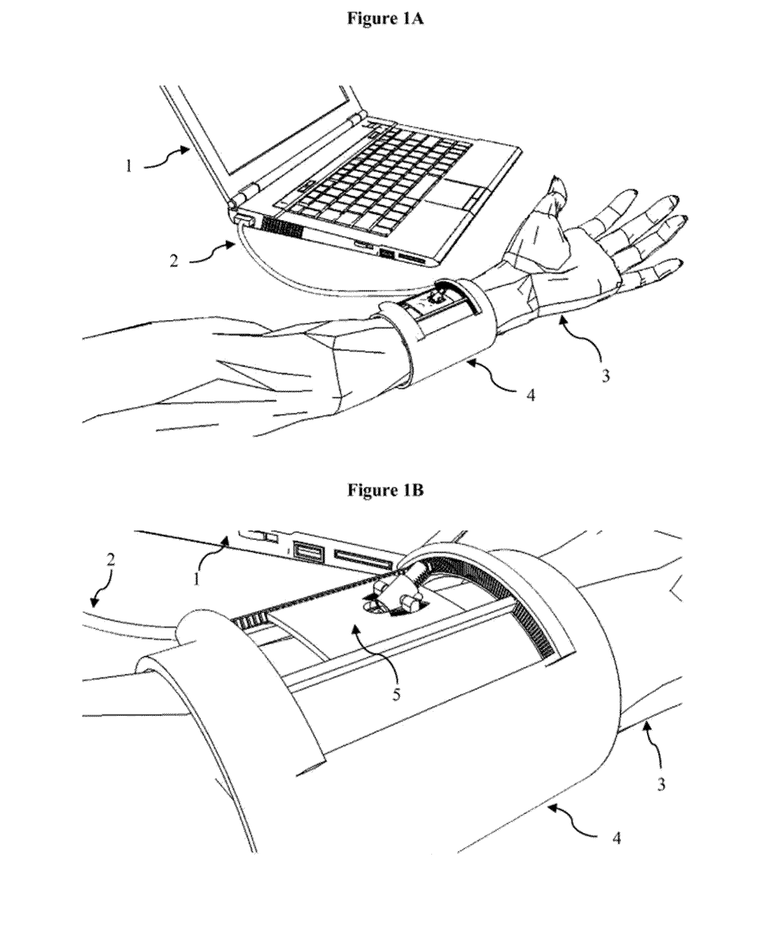

Based on the light reflected from the target vein, the light detector 7 generates an image, e.g., video image, of the target vein in the form of an electrical video signal. The enhanced video image signal is provided to a computer 1 through an interface cable 2, as shown in FIG. 1. The computer 1 captures still images from the image signal which may be saved in digital format on a digital storage device either in or connected to the computer 1. One skilled in the art would understand that various electronic storage devices, such as external hard drives and the like may be utilized in the present invention.

In one embodiment, acoustic imaging will be employed using 12 MHz ultrasound probe and ultrasound electronics to enable A-, B- and C-scans on the user interface. The acoustic ultrasound imaging modality will include several electronic components: i) the transducer, ii) a signal pulser/receiver, iii) a digitizer, and iv) the processing unit, which will be incorporated within the computer software. Other embodiments use transducers with different frequencies, in the range of about 5 MHz to about 14 MHz.

The transducer is designed to be moved over the surface of the appendace, wherein it is pulsed and receives echoes from the surface. This process is repeated many times a second at >50 kHz to generate the signal. The transducers are built around piezoelectric ceramics that vibrate at ultrasonic frequencies when a voltage is applied, and generate voltages when vibrated. The transducers are packaged within housings, and a single attachment interface allows interchanging different transducers to the overall automated vessel puncture system depending on the specific clinical application.

In certain embodiments of the present invention, multiple transducers will be combined using matrix or multiplex switches.

The pulser/receiver is connected to the transducer through a cable, and provides the high-voltage pulse required by the ultrasonic transducer as well as signal conditioning before the analog signal is passed to the digitizer. For use within an automated test system, the pulser/receiver is computer programmable via a standard PC bus such as RS232 or USB. Pulse voltage level, pulse repetition frequency, damping, band pass filtering settings, and several other parameters are established based on electronic requirements.

The digitizer converts the echo waveforms returned by the ultrasonic transducer into digital information using an analog-to-digital converter (ADC). A sample rate 10 times higher than the resonant frequency of the transducers is used; specifically, with a transducer that has a resonant frequency of 5 MHz, as is the case in one embodiment, 50 MS/s is required to accurately represent the shape of the signal. However, for applications that require less amplitude and echo-timing accuracy, four to five times the resonant frequency is acceptable, as is the case in another embodiment. The digitizer is employed in the described ultrasound imaging system as a timing slave while the pulser/receiver acts as the master. A high speed bus is incorporated to handle the large sample data and high frequency.

In certain embodiments of the present invention, a motion controller is incorporated to gather multiple data points with one transducer. In the B- or C-scan mode, the controller moves the ultrasonic sensor over the appendage surface to create a 3D surface map. The motion controller will comprise servo and/or stepper motor control, multi-axis control, and a position feedback interface. A combination of position, velocity, and acceleration triggering, which are commonly referred to as breakpoints, is used to ensure the test is accurate and repeatable. Also included ability to share motion breakpoints with other I/O without external cabling, as with PXI, guarantees all I/O lines are synchronized in a repeatable fashion.

The motion controller, digitizer, and pulser/receiver must operate as one tightly timed unit during the test to ensure accuracy of results and brevity of test time. The pulser/receiver can act as the master timebase of the system or can act as a slave to the digitizer or motion controller. In the applications where motion control is implemented, it is typically the slowest part of the system and, for that reason, acts as the master timebase.

The frequency of the transducer is chosen based on the required sensitivity and depth of penetration. The higher the frequency, the better the sensitivity but the less the depth of penetration.

Ultrasonic application software will be incorporated to drive the ultrasound imaging system, and will comprise I/O and analysis algorithms to form the interface. The application software comprises three basic parts: acquisition/control, analysis, and presentation. Analysis algorithms are incorporated into the computer processing system, wherein a number of filters are employed including peak detection, computation of distances based on material properties, wave rectification, statistical analysis, fast Fourier transforms (FFT), level crossing, frequency based modulation, and lock in amplification.

Several ways to look at ultrasonic test data ranging from TOF to surface scan: The most common are referred to as A-, B- and C-scans. The TOF scan, or A-scan, is analogous to the display on an oscilloscope, which shows voltage amplitude versus depth. The depth is calculated by multiplying the speed of sound through the medium by the time of flight.

Photoacoustic imaging is another method of visualizing subcutaneous blood vessels that may be utilized as an imaging modality within the present automated vessel puncture device. Photoacoustic imaging, which is based on the photoacoustic effect, uses the combination of high ultrasonic resolution with good image contrast because of differential optical/rf absorption. When compared with fluorescence imaging, in which the scattering in tissues limits the spatial resolution with increasing depth, photoacoustic imaging has higher spatial resolution and deeper imaging depth, because scattering of the ultrasonic signal in tissue is much weaker. When compared with ultrasound imaging, in which the contrast is limited because of the mechanical properties of biological tissues, photoacoustic imaging has better tissue contrast, which is related to the optical properties of different tissues. In addition, the absence of ionizing radiation also makes photoacoustic imaging safer than other imaging techniques, such as computed tomography and radionuclide-based imaging techniques.

In certain embodiments of the present invention, a 50 MHz pulsed laser diode array in the wavelength range of 760 to 1060 nm is used as the light source and an ultrasound transducer is used as the detector. In a second embodiment, a continuous wave laser diode array in the 760 to 1060 nm wavelength range serves as the light source. In the continuous wave embodiment, the power of the laser diode is modulated to about 2.5 MHz with 100% modulation depth by applying a 10 V sinusoidal signal from a function generator to the laser bias. In one embodiment, light from the laser diodes are delivered through an optical fiber. Analysis algorithms such as peak detection, wave rectification, statistical analysis, FFT, level crossing, and frequency modulation, and lock in amplification are incorporated into the computer processing system.

More than one method of imaging may be integrated for vessel detection. In certain embodiments, near infrared optical imaging in the wavelength range between about 730 nm to about 1060 nm will be used to image superficial vessels up to 4 mm in depth, while acoustic ultrasound or photoacoustic imaging will be used to image vessels below 4 mm. In other embodiments, photoacoustic imaging will be utilized in conjunction with the near infrared optical imaging modality and the acoustic ultrasound modality.

As shown in FIGS. 1A and 1B, a preferred embodiment of the present invention is contemplated wherein the imaging system is housed within imaging housing unit(s) 5 that houses the components of the imaging system 4. The present invention is not limited to this specific set-up and other methods for securing the imaging system to the self-contained, automated puncturing device are contemplated.

In certain embodiments, the imaging housing unit(s) may be disengaged from the overall device structure to be used as standalone imaging system(s). In such cases, the software developed for the automated vessel puncture device will also have modalities that enable standalone use.

### Automated Robotic End-Effector Unit

In preferred embodiments, another component of the self-contained, automated venipuncture device of the present invention is an automated robotic end-effector unit that provides robotically controlled needle motion and which is capable of robotically guiding the needle into a target vessel designated by the computer or operator.

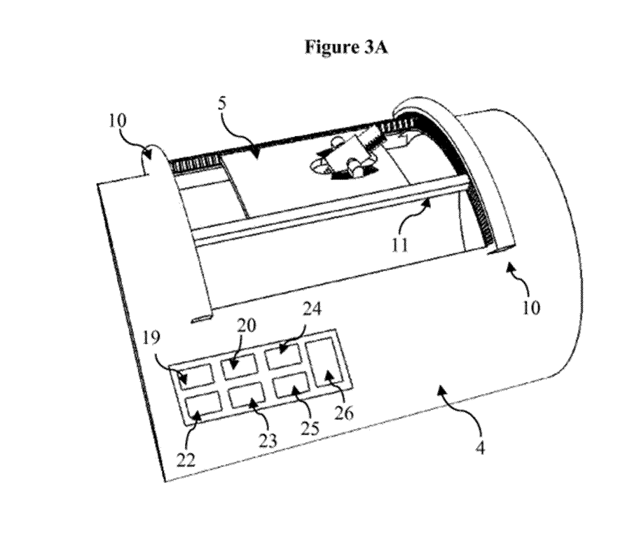

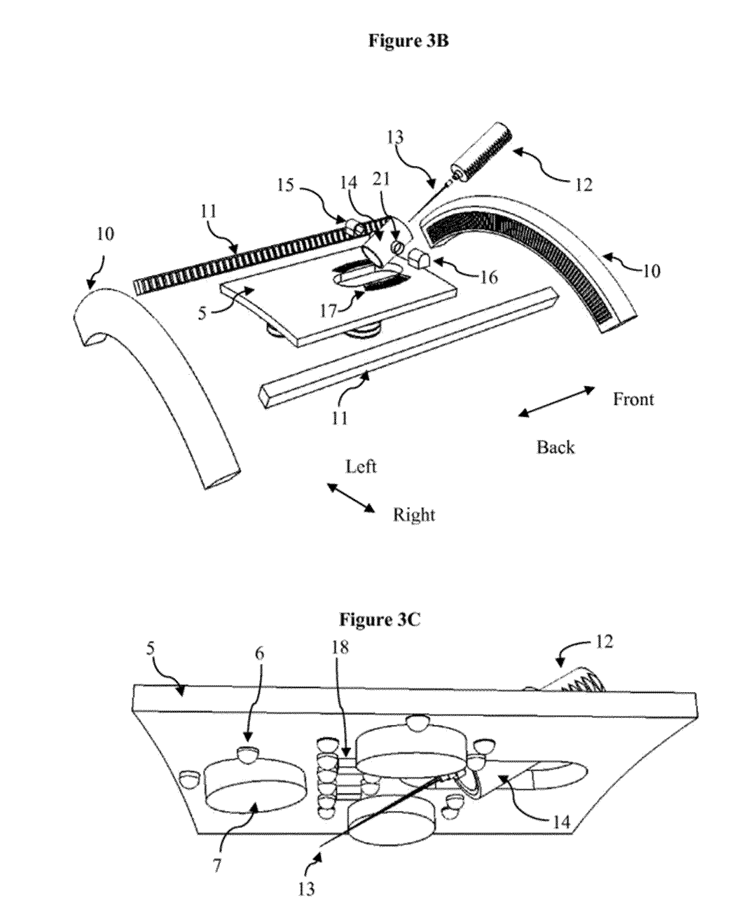

A representative automated robotic end-effector unit of the self-contained, automated venipuncture device of the present invention is shown in FIGS. 3A, 3B, and 3C. In certain embodiments, the robotic effector comprises 4 or 5 degrees of freedom (DOF), whereas in other embodiments, the robot comprises 6 DOF. In the 6 degree of freedom system, a 3 DOF gantry system is combined with a 3 DOF manipulator arm that carries the needle.

The gantry system provides synchronous position control of three linear stages with integrated controllers, achieving error tolerances in the micrometer range. The lower-axis master-slave dual X stage, upper-axis Y stage, and vertical Z stage use coupled leadscrews to reduce backlash and allow ample workspace underneath the gantry—enough for the system to scan the entire length of the arm and for the needle to access the major veins of the forearm and ACF.

The 3 DOF micromanipulator arm and stereo cameras are mounted onto the Z stage. In one embodiment, the arm measures consists of two rotational microsteppers controlling the horizontal and vertical angles of insertion (pitch and yaw, respectively) as well as a linear actuator controlling the forward motion of needle insertion. The cameras are rigidly mounted to the device above the manipulator.

The overall system must also include a mechanism for holding the human's body, e.g., limb, still and in place during the procedure. By providing a support, the device can improve the comfort of the procedure and reduce movement. Numerous methods for providing support may be utilized. In a preferred embodiment of the invention, an air-inflatable cuff-like structure enclosed around the appendage, similar in nature to a blood pressure cuff, that is adapted to different patient sizes. The support may furthermore be tightened either above or below the site of cannulation, to serve as a tourniquet. A method is included for preventing the subject's appendage from coming in contact with the mechanical and electronic components of the device to improve device safety. Finally, all components of the support that may come in contact with the subject may be disengaged from the remainder of the device for the purpose of sterilization.

To automatically guide the needle during the cannulation routine, the robotic end-effector may utilize a combination of a guidance system based on a derived three-dimensional (3D) coordinate map of the target vessel, and in some embodiments also utilize a guidance system based on haptic or force feedback. Both systems alone have advantages and disadvantages, but if used in compliment can provide an optimal system vascular access. The haptic system alone is limited in the fact that it does not take into consideration the depth penetrated within the system, only the fact that the vein has been punctured. The 3D coordinate map based guidance system will validate that the needle is in the correct location, that it has entered the vessel, and that a certain penetration depth has not been exceeded. This imaging system is only one level of safety protection. In order to ensure a robust safety mechanism for the device, both systems will be employed in tandem to ensure safety.

The three-dimensional (3D) imaging technology of the present invention can be used to automatically and accurately guide a needle to a location of the target vessel. Actual insertion of the needle into the skin and into a vessel is a dynamic process due to the elasticity of tissue. Stretching and deformation of the skin will result in effects not anticipated or compensated for by a system based on visualization alone. Therefore, haptic or force feedback is used in certain preferred embodiments to account for these effects.

Tactile or force based imaging is incorporated in certain embodiments of the present invention to either image blood vessels prior to cannulation, or provide a method of feedback during the needle insertion process. Recent advances in industrial transducer design have allowed for commercially available miniature force sensors, such as the Nano model line manufactured by ATI, to demonstrate sub-gram-force resolution up to 100 times that of human touch. In one embodiment utilizing force sensing, a 6-axis force transducer (ATI Nanol7) is coupled to the base of the needle shaft. Other embodiments utilize 1-axis or 3-axis transducers.

A computer model that relates the forces at the needle base to displacements of the tip is combined with the force sensor to provide needle guidance during insertion. In conjunction with the model, the described sensor measures the force acting on the needle tip in the axial direction, the friction forces acting on the shaft wall in the axial direction, and the bending forces acting on the shaft wall in the normal direction. In one embodiment, the described transducer has a force resolution of 0.78 mN along each orthogonal axis and a bandwidth of 10 kHz. Cylindrical indenters are be fitted to the tip of the force sensor to apply the displacement stimuli without introducing significant contact nonlinearities. Reaction force data samples are time-coded at 1000 fps using a 16-bit analog-to-digital converter and analysis software.

The kinematic computer model predicts needle and tissue deformations due to reaction forces during needle insertion into a blood vessel. The computer model describes the nonholonomic kinematics of a needle manipulation Jacobian. The Jacobian relates needle tip motion from needle base motion in conjunction with a tissue finite element model. The computer model accounts for multiple tissue types and incorporates the elastic and viscoelastic properties of the skin and vessel as input parameters to the overall kinematic model. Overall the model serves as a method that predicts needle bending, needle tip displacement, and vessel displacement from normal and shear forces acting on the needle for the purpose of blood vessel puncture within the automated vessel puncture device of the present invention.

Utilizing the self-contained, automated venipuncture device of the present invention, when the needle is actually inserted into the skin, and punctures the target vessel wall, force and position profiles are generated that are sufficiently distinct to implement automatic needle withdrawal, preventing an overshoot of the needle.

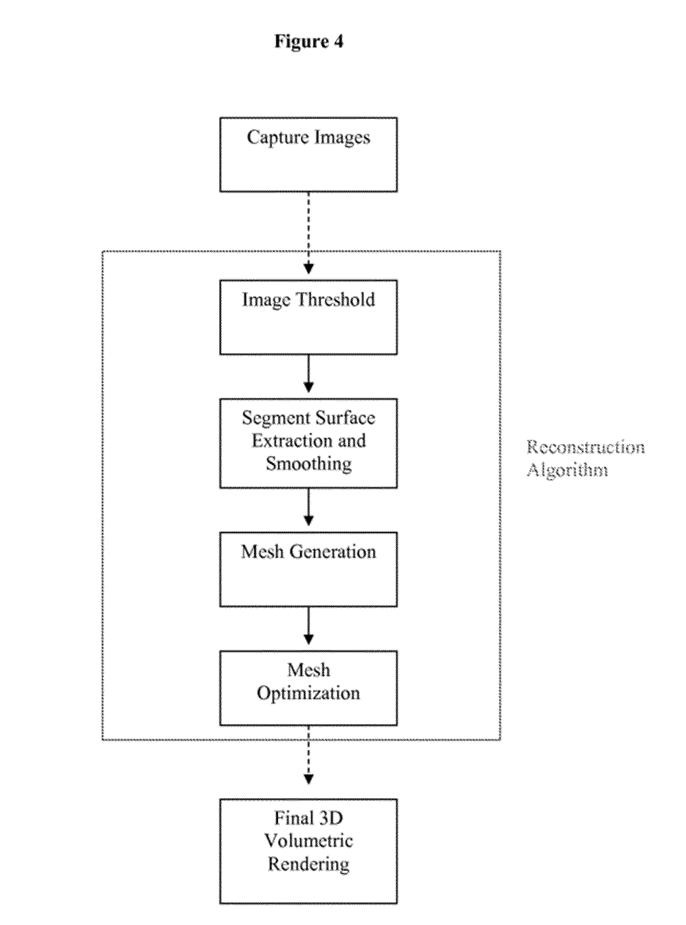

In certain embodiments of the present invention, one governing algorithm that may be utilized to control the position and injection of the needle 13 (FIGS. 3B, 3C) into the target vessel is functionally diagrammed in FIG. 5. A first processor 19 (FIG. 3A) is provided for calculating a relative needle target puncture position using the 3D volumetric image provided by the image reconstruction program is first used to compute a relative target position for the needle (FIG. 5, Step 1). A position sensor 18 (FIGS. 2A, 2B, 3C) is provided for identifying the absolute distance description of a device reference point from the target vessel (FIG. 5, Step 2). A second processor 20 (FIG. 3A) is provided for calculating the absolute target spatial position for the needle by adjusting the relative target position from the first processor 19 by the absolute distance obtained from the position sensor 18 (FIG. 5, Step 3). A second position sensor 21 (FIG. 3B), within the device carrier housing 14 (FIG. 3B), is provided for identifying the current position description of the needle device carrier 12 (FIG. 3B). A third processor 22 (FIG. 3A) is provided for feedback control of the needle device carrier 12 with respect to the absolute target spatial position for the needle provided from the second processor 20. The third processor 22 stops the needle carrier 12 when the spatial position from the second position sensor 21 coincides with the absolute target spatial position for the needle provided from the second processor 20 (FIG. 5, Step 4) Finally up and down movement of the needle 13 is controlled by a fourth processor 23 (FIG. 3A), which adjusts the angle of the needle 13 through servo motor 16 (FIG. 3B) (FIG. 5, Step 5).

First processor 19, second processor 20, third processor 22, and fourth processor 23, are computational units which can be software or hardware modules arranged separately or in any appropriate combination as part of a computer 1. In addition these processors could also be subroutines within a piece of software contained in a computer 1.

The movement of the needle device carrier 12 is driven by a set of servo motors 15, 16, contained within the needle device carrier housing 14. Left to right coarse adjustment (shown by the arrow in FIG. 3B) is driven by servo motors within gear railing 10. Front to back movement (shown by the arrow in FIG. 3B) is driven by servo motors within the gear railing 11. Fine left to right movement is driven by servo motor 15. Fine up and down movement is driven by servo motor 16.

Injection of the needle 13 is driven by servo motors within the needle device carrier housing 14.

After the medical procedure of interest is completed, a signal from a fourth processor 23, will reverse the servo motor within 14, to withdraw the needle 13 from the target vessel, and subsequently return the needle device carrier 12 to the starting position.

### Computer Component

The computer component of the present invention performs several discreet functions. These include (1) controlling the light source (e.g., LEDs)/light detector array (e.g., photodetectors); (2) creating a three-dimensional (3D) map of the target vessel position; (3) controlling the motion of the automated robotic end-effector unit; and (4) receiving feedback from the end-automated robotic end-effector unit for purposes of generating force and position profiles, and applying this feedback by adjusting the amount of force applied to the needle to penetrate the skin and vein of the human.

Any commonly available personal computer may be used for these purposes. The computer must have a physical interface to both the light source/light detector units and to the automated robotic end-effector unit. The computer must have the capability of turning on and off various light sources and reading the results from the light detectors. In addition, the computer must be capable of providing commands to the automated robotic end-effector unit and reading feedback signals from there. Additionally, the computer must be capable of generating the three-dimensional (3D) maps and the force and position profiles. One with skill in the art will realize that there are many ways of implementing software to perform these functions, and the actual arrangement and architecture of that software is not the subject of this invention.

In certain embodiments, the computer will utilize a software program for reconstructing a three-dimensional model from the images housed in a computer interface. The same software may also be utilized for evaluating the three-dimensional images and guiding the robotically driven needle.

In certain other embodiments, a small, special purpose ASIC (application-specific integrated circuit) may also be utilized in place of the computer and may be integrated into the device. Additional, hardwire logic, gate array and state machine technologies can also be utilized in place of the computer.

As a means to provide clinical decision support and warrant operational safety, it is a major goal that the automated vessel puncture device of the present invention incorporates methods to automatically select an optimal vein, track the vein in real-time, and determine the manner of injection based on updated visual information about the position and orientation of the selected vein. To automatically determine the location and manner of needle insertion into the vessel, it is a major emphasis of the device to simulate the complex deductive strategies utilized by clinicians, in which their recognition of visual cues is augmented with prior knowledge of anatomical landmarks. To this end, a vessel selection and tracking algorithm is incorporated into the control software. The algorithm considers multiple factors (for example, the depth, rigidity, and “fullness” of the vessel) and the decision is predicated on correctly identifying the major veins of the forearm and elbow. The cannulation site is then tracked as the clinician guides the needle into the vessel.

In a preferred embodiment, the vessel selection software employs graph theory techniques to identify and label the vessels of the subject's appendage based on their anatomical structure and relative position on the appendage. The algorithm, utilizing graph theory, comprises three major steps: 1) Construct a “ground-truth” skeleton graph by identifying the most conserved structures across a large population of humans to serve as the template in the subsequent steps of the algorithm; 2) Identify veins from processed images and extract the 3D medial axis skeletons from the reconstruction of stereo image pairs; 3) Apply transformations to rescale and align each skeleton based on the positions and orientations of the elbow and wrist as determined from the 3D forearm reconstruction; 4) Convert the skeletons into graphs having the same basic structure as the ground truth graph; 5) Compare the similarity of each branch in the skeleton graph to each branch of the ground truth graph; 6) Using predetermined weight parameters as inputs to the final decision system, identify and rank potential cannulation sites based on their measured suitability for puncture. Herein, by limiting the set of possible positions for needle insertion to these targets, the likelihood of diversion error during the vessel tracking phase is reduced.

To track the selected vein in real time as the needle is inserted, an image based tracking algorithm is incorporated into the control software that uses frame-by-frame global matching of images produced by one or more cameras and/or the signal from one or more ultrasound transducer. The tracking technique will modulate the density of feature points used based on their suitability for cannulation as determined by the vein selection algorithm described above. Thus, feature points will be dense around veins, especially so for those clinically appropriate for puncture, and sparse in the background. Accordingly, the precision and stability of the frame match will be maximal in those areas where precision and stability are most needed. In one preferred embodiment, the tracking algorithm will apply belief propagation, where the “quality” of a match at each pixel is updated via recursive message-passing to and from its neighboring pixels. The method is designed to minimize computational expense in two ways: 1) the aforementioned use of selective feature points; and 2) the use of hierarchical approach, in space and in depth, to refine messages at a course level before passing them to a finer stage. The tracking software is able to operate at a speed of 16 frames per second.

### Safety Feedback System

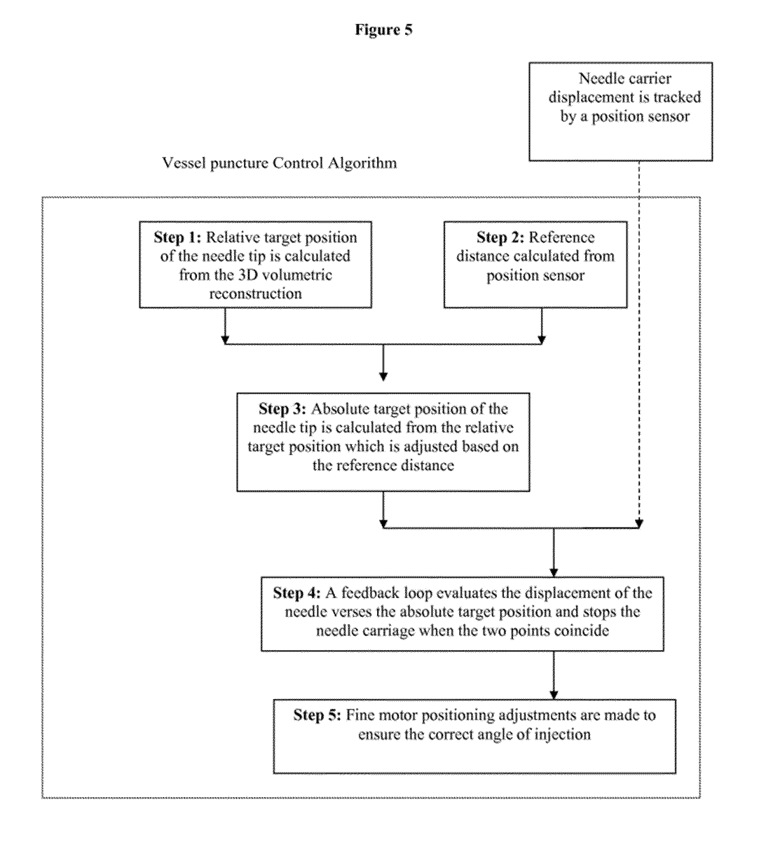

A pressure sensor contained within the needle device carrier housing 14, is responsible for needle injection transfers pressure readings to a first processor 24. The first processor 24 computes a change in applied force over time. A second processor 25, monitors the change in applied force over time and will switch off the servo motor, within the needle carrier housing 14, after an increase in pressure is observed, FIG. 6.

A secondary safety system is also included through the imaging system and 3D reconstruction algorithm. While venipuncture is taking place, the imaging system and reconstruction algorithm are working in real time and will determine the penetration depth of the needle 13 into the target vessel. A third processor 26, will integrate the penetration depth data with the pressure sensor data from the second processor 25 and will ensure that the servo motor, within the device housing 14, is switched off either after the aforementioned change in pressure is observed or the correct depth is penetrated.

Real time needle tracking is incorporated as an additional measure of safety. The needle is segmented and the needle tip is identified as the front most point of the segmented object. The predicted 3D position of the needle is then compared to the position of the needle robot manipulator. Agreement between the needle position derived by image tracking and the position given directly by the motors, to within 0.25 mm, is considered acceptable. A continuous period t of disagreement (here, set to t>1 sec) results in the termination of the runtime loop and a recommendation by the device for recalibration.

The feedback guidance methods comprise the steps of: i) calculating a relative target position of the needle tip utilizing a three-dimensional reconstruction of the blood vessels; ii) calculating a reference distance of the needle tip utilizing a position sensor located on the imaging system; iii) calculating the absolute target position of the needle tip based on the relative target position of step i) that is adjusted based on the reference distance of step ii); iv) tracking the displacement of the needle device carrier by the position sensor; v) evaluating the displacement of the needle verses the absolute target position utilizing a feedback loop, wherein needle placement is stopped when the needle displacement and absolute target position coincide; vi) ensuring the correct angle of injection utilizing fine motor positioning adjustments, such that venipuncture to an optimal vein is provided.

Additional safety mechanisms built into certain preferred embodiments of the device include mechanical safety features. Mechanically, all axes of the robot are finely pitched to minimize backdrivability and ensure low maximum speeds. Rotational motion is gravity balanced, ensuring that the robot will stop during emergencies, crashes, or loss of power.

A watchdog protocol is used to activate fail-safe circuitry when a fault is detected. When activated, the fail-safe circuitry forces all control outputs to safe states. The watchdog is also used to monitor the activity of the main control program and the electrical state of the system. The watchdog triggers the control program to update the register of a timer at a cycle rate of 10 Hertz. Failure to trigger causes an emergency power shutdown.

A safety-release mechanism is also incorporated that allows the needle to be released manually when the vein target is reached actuation is no longer needed, or in cases of emergency where the needle must be immediately detached. The hinged attachment piece is fabricated from a solid plastic material, is low cost and disposable, can be sterilized, and can be easily manufactured to accommodate standard needle sizes.

### Methods of Three-Dimensional Imaging

Another embodiment of the present invention is directed to methods of conducting fully automated venipuncture in a human. The method combines the self-contained, automated venipuncture device of the present invention together with one or more vessel imaging techniques to generate a three dimensional map of subcutaneous vessels in real time. Methods of imaging include visible or infrared optical imaging, acoustic imaging, photoacoustic imaging, and tactile and force based imaging. A combination of these methods can also be utilized and integrated with the computer.

Optical imaging methods traditionally provide a two dimensional representations of the vessels. By combining multiple visible or infrared images of the vascular network, one can generate a three dimensional representation of the vessels. This will far exceed current techniques of 3D visualization in efficiency, time and cost. The resultant 3D representation of the vessel will subsequently be used to provide spatial position cues to an automated venipuncture device.

Two dimensional images are processed and vessels observed in the images are emphasized based on their intensity and geometric characteristics. Blood vessels, being tubular, exhibit highly negative curvature in one direction and near-zero curvature in the orthogonal direction, as well as dark intensities due to hemoglobin absorption. These characteristics are exploited in a series of image processing steps to highlight vessels and reduce the presence of artifacts and noise.

One preferred method of vessel enhancement involves the use of the information stored in the image Hessian matrix. The eigenproperties of the Hessian can thus be used to define a measure of “vesselness” from similarities to a generalized cylinder model using the second-order approximation of the image function f(x);

ƒ″(x)=ƒ(x₀)+(x−x₀)T^{v}ƒ₀+0.5(x−x₀)T^{v2}ƒ₀(x−x₀),

where ^{v}ƒ₀ and ^{v2}ƒ₀ denote the gradient vector and Hessian matrix at x₀, respectively. Letting the eigenvalues of ^{v2}_{ƒ}₀ be λ₁, λ₂ (λ₁>=λ₂) and their corresponding eigenvectors be e₁ and e₂, it results that λ₁ gives the maximum second derivative value at each pixel, and e₁ the corresponding direction while λ₂ gives the second-derivative value in the orthogonal direction, and e₂ is its direction. The images acquired by the device present vessels as dark tubular structures against a light background; accordingly, the algorithm looks for structures with negative λ2. To summarize the geometric characteristics an ideal tubular structure in a 2D image, we have:

|λ_{⊥}|≈0 |λ₁|<<|λ₂|R_{B}=λ₁/λ₂ λ₂<0

A distinguishing property of background pixels is that the magnitude of the derivatives (and thus the eigenvalues) is small. To account for this, we use of the norm of the Hessian, given by S, which will be low in the background. In regions with high contrast compared to the background, the norm will be larger since at least one of the eigenvalues will be large. Thus the vessel enhancement algorithm in the preferred embodiment finally utilizes a geometrical model that combines the above parameters to give a final measure of “vesselness”,

$${V}_{o} ( s ) = \{ \begin{matrix}0 & if {λ}_{2} < 0 \\ exp ( - \frac{{Rp}^{2}}{2 {β}^{2}} ) ( 1 - exp ( - \frac{{S}^{2}}{2 {σ}^{2}} ) ) & \end{matrix}$$

where β and c are weighting factors for RB and S, respectively. In this way, depending on the internal characteristics of the real time images, objects that correspond highly with the geometrical model of blood vessels will be highlighted while objects that correspond poorly will be suppressed.

In addition to enhancement, the methods of the present invention allow for processed images to furthermore be combined into a three-dimensional (3D) reconstruction of the target vessel. This process will be executed by a computer program contained within the computer 1.

In one embodiment, the imaging technique of Diffuse Optical Tomography is utilized to generate the 3D representation of the vessels. Presently, diffusion optical tomography is a widely utilized optical image reconstruction tomographic technique. Examples of references which disclose this technique include: U.S. Pat. No. 5,813,988 to Alfano et al. entitled “Time-Resolved Diffusion Tomographic Imaging In Highly Scattering Turbid Media,” which issued Sep. 29, 1998; W. Cai et al., “Time-Resolved Optical Diffusion Tomographic Image Reconstruction In Highly Scattering Turbid Media,” Proc. Natl. Acad. Sci. USA, Vol. 93 13561-64 (1996); Arridge, “The Forward and Inverse Problems in Time Resolved Infra-red Imaging,” Medical Optical Tomography: Functional Imaging and Monitoring SPIE Institutes, Vol. IS11, G. Muller ed., 31-64 (1993); and Singer et al., “Image Reconstruction of Interior of Bodies That Diffuse Radiation,” Science, 248: 990-3 (1993), all of which are incorporated herein by reference.

In another embodiment of the invention, stereoscopic imaging with two or more cameras is employed to generate the 3D vessel map. In the stereo imaging method, multiple cameras are aligned and calibrated with respect to their relative positions in space as well as their internal geometries. Once calibrated, images of the subcutaneous vessels, taken by both cameras, are analyzed and matched to one another The spatial offsets of each image with respect to the others are used as information to reconstruct the three dimensional structure of the vessels.

In embodiments of the present invention wherein acoustic or photoacoustic imaging methods are incorporated, the depth information inherently provided by the systems are processed and utilized to directly generate the volumetric 3D map of the vessels. Sound waves being sent down into the subject's appendage and reflected back, either directly in the case of the 2D scan or at different angles in the case of the 3D volumetric scan. The returning echoes are processed, resulting in a reconstructed three dimensional volume image. 4D scans are similar to 3D scans, with the difference associated with time: 4D allows a 3-dimensional picture in real time, rather than delayed, due to the lag associated with the computer constructed image.

In one embodiment of the invention, 3D ultrasound is acquired with 2D ultrasound probes using a three-step process: 1) a positioning sensor is attached to the probe; 2) a reconstruction of a 3D volume is performed into a regular voxel grid; 3) processing and reconstruction algorithms are employed for performing 3D reconstruction based on 2D images. Such algorithms include voxel-based traversal methods, voxel-based interpolation methods, pixel based methods, function-based methods, 3D kernals, and pixel nearest neighbors bin filling methods.

Integration with Point of Care Analytical Applications

The present invention is also directed to integrating the self-contained, automated vessel puncture device as a kit, or a modified device to include analytical assays. These point of care assays include, but are not limited to: 1) glucose monitoring; 2) determination of pregnancy/ovulation; 3) measurement of coagulation/PT; 4) fecal occult blood; 5) determination of drugs of abuse; 6) detection of H. pylori; 7) detection of HIV; 8) monitoring of cholesterol levels.

For these types of applications, blood can be withdrawn from a target vessel of a human utilizing the self-contained, automated vessel puncture device of the present invention and then introduced into a point of care diagnostic assay.

1. An automated system for vascular imaging and access, comprising:
an imaging system for providing continuous and real-time imaging of blood vessels, said imaging system comprising at least one of optical, acoustic, photoacoustic, or tactile imaging;
image processing software for generating a continuous and real-time three-dimensional (3D) computer model of the blood vessels based on the imaging system, and selecting an optimal vessel target based on visual and anatomical information for inserting a needle into the selected vessel target during a cannulation routine on a human subject;
a robotic effector comprising a needle, a needle attachment unit, and a needle actuation system that positions the needle at the selected vessel target located by the image processing software and moves the needle toward and insert the needle into the selected vessel target; and
a computer connected to the imaging system and robotic effector, said computer directing information continuously and in real-time to and from the imaging system, image processing software, and robotic effector in order to autonomously adjust the position and orientation of the needle with respect to the selected vessel target during the cannulation routine.

2. The automated system of claim 1, wherein the robotic effector is adapted for placement on an appendage of a human or animal subject.

3. The automated system of claim 1, wherein the robotic effector can withdraw fluid from the selected vessel target or deliver fluid through the selected vessel target.

4. The automated system of claim 1, wherein the imaging system is a portable or tabletop device, capable of either being mounted onto a subject's appendage or being placed onto a stationary unit.

5. The automated system of claim 1, wherein the imaging system comprises at least one of:
at least one light emitting source having a wavelength emission range within the visible or infrared spectrum and one or more optical detectors; or
at least one ultrasound transducer, the acoustic signal being transmitted and received through the at least one ultrasound transducer and converted from analog to digital form for image formation and processing; or
at least one visible or near infrared optical light source and at least one ultrasound transducer, the photoacoustic signal being transmitted through the at least one optical light source, received through the at least one ultrasound transducer, and converted from analog to digital form for image formation and processing.

6. The automated system of claim 5, wherein the imaging system further allows for at least one of thermal imaging, spectroscopic imaging, diffuse optical tomography imaging, ultrasound Doppler imaging, ultrasound color Doppler imaging, 3D/4D ultrasound imaging, photoacoustic Doppler imaging, photoacoustic color Doppler imaging, or 3D/4D photoacoustic imaging.

7. The automated system of claim 5, wherein the imaging system is adapted to illuminate structures in addition to blood vessels, the additional structures comprising one or more of arm length, arm thickness, anatomical markings, markings on the skin surface, signs of infection, signs of fluid leakage, or nerves.

8. The automated system of claim 5, wherein the imaging system is further adapted to differentiate between veins and arteries.

9. The automated system of claim 5, wherein the imaging system is enhanced with at least one of diffusive filters, polarizing filters, spatial filters, coherence-based filters, wavelength or frequency-based bandpass filters, or electronic analog filters.

10. The automated system of claim 5, wherein the imaging system can be adjusted to maximize imaging quality on a per patient basis.

11. The automated system of claim 5, wherein the imaging system is contained within an imaging housing unit.

12. The automated system of claim 12, wherein the imaging housing unit can be temporarily disengaged from the robotic effector, the imaging system then being used as a standalone imaging system to visualize blood vessels or other structures.

13. The automated system of claim 11, wherein the imaging system is contained within the imaging housing unit and when disengaged from the robotic effector, can remain in communication with the computer.

14. The automated system of claim 1, wherein the image processing software processes and maps a three-dimensional (3D) computer model of blood vessels continuously and in real-time, via one or more of the following:
(i) increasing the visibility of vessels or decrease the amount of artifacts and noise in determining an optimal vessel target for cannulation;
(ii) computing the depth of the vessels and building a three-dimensional model of the vessels;
(iii) determining an the optimal vessel target for cannulation and the optimal needle orientation relative to a the-selected vessel target;
(iv) tracking the position and orientation of a the selected vessel target and the needle tip as the needle is moved toward and inserted into the selected vessel target;
(v) relaying information about the position and orientation of the selected vessel target and the needle based on the vessel and needle tip positions as the needle is moved toward and inserted into the selected vessel target.

15. The automated system of claim 15, wherein detected veins are automatically labeled and the selected vessel target is automatically determined based on at least one of the size and anatomical structure of the blood vessels, the quality of the image at each location, the particular needle and application, and the subject's medical information.

16. The automated system of claim 15, wherein the 3D position of the target vein and the 3D position of the needle tip are computed in real time, and wherein the relative distance between the 3D positions of the target vein and the needle tip is computed as the needle is inserted, and wherein the needle insertion is halted when the distance is zero.

17. The automated system of claim 15, wherein the pose of the needle is computed in real time, and wherein the correct angle of injection is ensured utilizing fine motor positioning adjustments.

18. The automated system of claim 15, wherein at least one of optical, mechanical, magnetic, or potentiometric sensors are coupled to at least one motor actuator in order to collect signals continuously and in real-time to indicate the rotational position and motion of the at least one motor actuator.

19. The automated system of claim 15, wherein at least one force sensor is coupled to the needle in order to collect signals continuously and in real-time to indicate mechanical forces acting on the needle as the needle is inserted.

20. The automated system of claim 15, wherein the feedback guidance methods additionally comprise a computer model of the mechanical interactions between the needle and the skin tissue, the computer model taking in signals from at least one feedback sensor and outputting the position of the needle tip and the target vessel continuously and in real-time.

21. The automated system of claim 1, wherein the needle is affixed to the robotic effector through the needle attachment unit such that the needle may be attached or detached manually or automatically before or after the cannulation routine.

22. The automated system of claim 21, wherein the robotic effector is capable of at least two of the following degrees of motion: i) horizontal motion along the length of the subject's appendage, ii) horizontal motion across the width of the subject's appendage, iii) vertical motion relative to the surface of the subject's appendage, iv) vertical angular rotation of the needle, v) horizontal angular rotation of the needle, vi) rotation of the needle along its axis, and vii) forward motion of needle insertion.
